(19) United States
(12) Patent Application Publication
(10) Pub. No.: US 2005/0049630 A1
(43) Pub. Date: Mar. 3, 2005

(54) Tourniquet device for single-handed operation

(76) Inventor: Robert Ambach, Oceanside, CA (US)

Correspondence Address: Mark L. Lorbiecki, Esq.;BLACK LOWE & GRAHAM PLLC, Suite 4800, 701 Fifth Avenue, Seattle, WA, 98104, US

(21) Appl. No.: 10/884,554

(22) Filed: Jul. 2, 2004

Related U.S. Application Data
(63) Continuation-in-part of application No. 10/125,808, filed on Apr. 18, 2002.

(51) Int. Cl. A61B 17/00

(52) U.S. Cl. 606/203

(57) ABSTRACT

A first belt extends between and through two opposing slip buckles that allow the belt to be pulled freely through them but only in respective directions which shorten the belt segment between the buckles. Each slip buckle includes a release which when operated allows the belt to slip freely through said each buckle in either direction. A second belt is affixed at one end to one of the slip buckles, the other end being connected to a mechanism by which a user can spool the second belt to selectively shorten its free length. The spooling mechanism is coupled to the other slip buckle, preferably by means of a fixed length belt, to complete a tourniquet loop. Preferably the spooling mechanism includes a ratchet mechanism operated by a lever in a pumping manner. Preferably the loop can be opened and closed.

### PRIORITY CLAIM

This application is a continuation-in-part of a utility application, Ser. No. 10/125,808, filed on Apr. 18, 2002 and entitled “Tourniquet Device for Single-Handed Operation. This reference incorporates the prior application and claims priority from its filing date, Apr. 18, 2002.

### FIELD OF THE INVENTION

This invention relates generally to medical treatment equipment and, more specifically, to first response medical treatment equipment.

### BACKGROUND OF THE INVENTION

This invention relates in general to the field of tourniquets and more particularly to a tourniquet device configure for operation with one hand.

Tourniquets are well known for temporarily restricting the flow of blood to a person's injured limb in order to prevent a serious loss of blood. If a person's arm is injured to the extent that significant blood is being lost, another person can generally fashion a tourniquet from available materials, such as a belt, rope, cord, or cloth, and apply it to the injured arm to restrict or stop the bleeding, allowing time to seek medical attention. However, when such an injury occurs where assistance is unavailable and the injured arm is incapacitated or severed, it is difficult, if not impossible, for the injured person to effectively apply a tourniquet to the injured arm. Depending on the rate of blood loss, the injured person may have only a very short time to stop the bleeding in order to survive. While the need for being able to self-apply a tourniquet to one's injured arm has existed through the ages, heretofore there has not been an effective solution.

### SUMMARY OF THE INVENTION

The tourniquet device of this invention enables application with one hand. With this invention, a person having a seriously injured arm can apply a tourniquet to the injured arm and tighten it with his free (uninjured) hand. This invention can actually be used to quickly and easily apply a tourniquet to any limb, not just an arm, and can of course be applied using two hands, but it is tremendously advantageous in all situations in which only one hand is available to apply a tourniquet. For example, a person with only one usable arm can apply this invention to another person's bleeding limb, especially in situations where the other person is either unconscious or otherwise unable to assist.

Other advantages and attributes of this invention will be readily discernable upon a reading of the text hereinafter.

A tourniquet loop adapted for single-handed operation comprising: a first loop segment being single-handedly adjustable in length for coarsely tightening the loop around a limb; and a second loop segment being single-handedly adjustable in length for further tightening the loop enough to at least restrict blood flow in the limb. Preferably the tourniquet loop can be opened and closed as needed.

In a preferred embodiment, the first tourniquet loop segment includes two opposing slip buckles; a first belt extending between and through the slip buckles, each slip buckle allowing the belt to be pulled freely through it in a direction which shortens the length of first belt between the slip buckles but preventing the first belt from traversing through it in the opposite direction; and a line means, gripable by a single hand, for pulling the first belt through the slip buckles in respective directions which shorten the length of first belt between the slip buckles. Preferably at least one slip buckle further includes a release which when selectively actuated allows the first belt to be pulled through said at least one slip buckle in either direction.

In a second preferred embodiment, the second tourniquet loop segment comprises a second belt affixed at one end to one slip buckle; a belt tightening mechanism, engaged with the other end of the second belt, by which a user can selectively shorten the length of second belt between the mechanism and said one slip buckle; and a coupling between the mechanism and the other slip buckle to complete the tourniquet loop. One embodiment of the belt tightening mechanism includes a rotatable spool onto which the second belt can be spooled; a lever by which the spool is turned in the direction of spooling the second belt; and a ratchet mechanism for allowing the spool to turn in the direction of spooling the second belt but preventing it from turning in the opposite direction.

A preferred embodiment of a slip buckle includes a base; a cylinder rotatable on an axis lateral to the first belt, the cylinder including a ridged eccentricity, the cylinder axis being so disposed in fixed relation to the base that the eccentricity can be rotated to impinge upon the first belt which extends between the base and the cylinder and when so rotated the eccentricity seizing and wedging the first belt against the base whenever the first belt is pulled oppositely to the direction which shortens the length of first belt between the slip buckles; and a bias which urges the cylinder to assume an angular disposition at which the eccentricity impinges upon the belt. Also preferably a slip buckle further includes a lever by which a user can rotate the cylinder against the bias to release the first belt and a release for allowing the second belt to be unspooled.

### BRIEF DESCRIPTION OF THE DRAWINGS

FIG. 1 is a pictorial perspective view of the invention;

FIG. 2 is a plan view of a preferred slip buckle according to this invention;

FIG. 3 is a cross-sectional view of the slip buckle of FIG. 2;

FIG. 4 is a pictorial view of a one-way locking cam that is a part of the slip buckle illustrated in FIGS. 2 and 3;

FIG. 5 is the first of four plan views illustrating operation of the invention (In this view, the invention has been loosely slipped over a person's injured arm represented in diamond hatched cross-section);

FIG. 6 is a plan view illustrating a first step in tightening the invention about the injured arm (In this view, the person has started coarsely tightening the invention about the injured arm by using his or her usable hand (not shown) to pull a tightening cord.);

FIG. 7 is a plan view illustrating a second step in tightening the invention about the injured arm (In this view the person has coarsely tightened the invention about the injured arm by further pulling on the tightening cord);

FIG. 8 is a plan view illustrating completion of a third step in tightening the invention about the injured arm (In this view the person has operated the spooling lever to suitably tighten the invention completely around the arm.);

FIG. 9 is a partial plan view illustrating an alternative embodiment in which the tourniquet loop can be selectively opened to facilitate application, and then re-closed to tighten it around a limb:

FIG. 10 is a pictorial view of a hook clasp, as illustrated in FIG. 9, useable to selectively open and close the tourniquet loop;

FIG. 11 is a pictorial view of a releasable fastener, useable to selectively open and close the tourniquet loop;

FIG. 12 is a plan view of a sliding slip buckle according to this invention; and

FIG. 13 is a cross-sectional view of the sliding slip buckle of FIG. 12.

### DETAILED DESCRIPTION OF THE PREFERRED EMBODIMENT

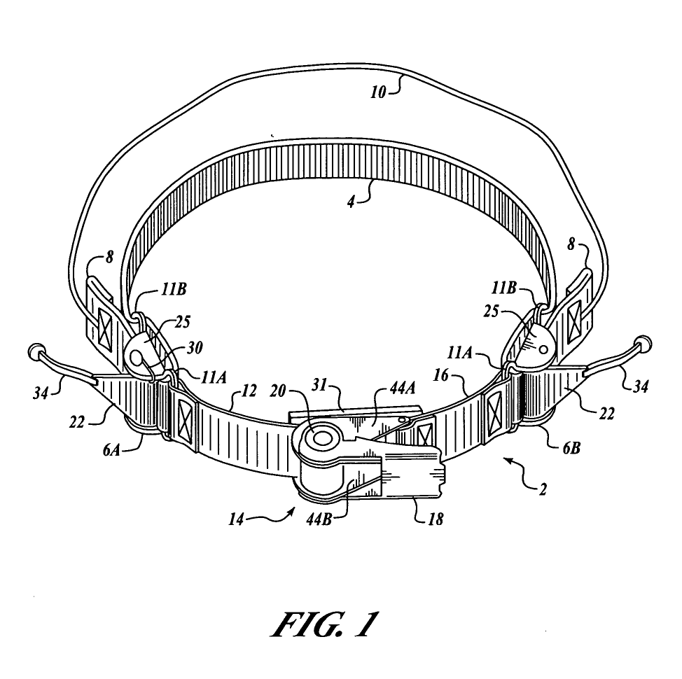

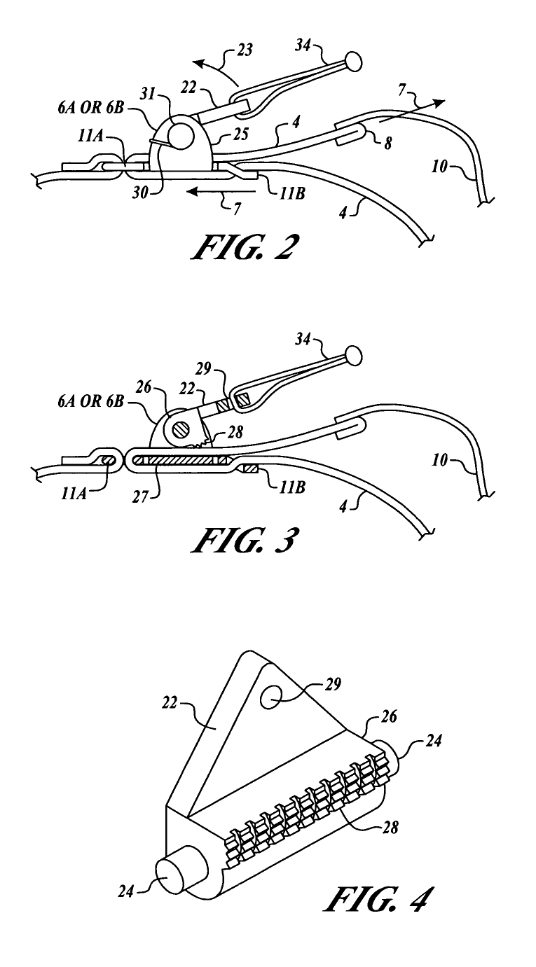

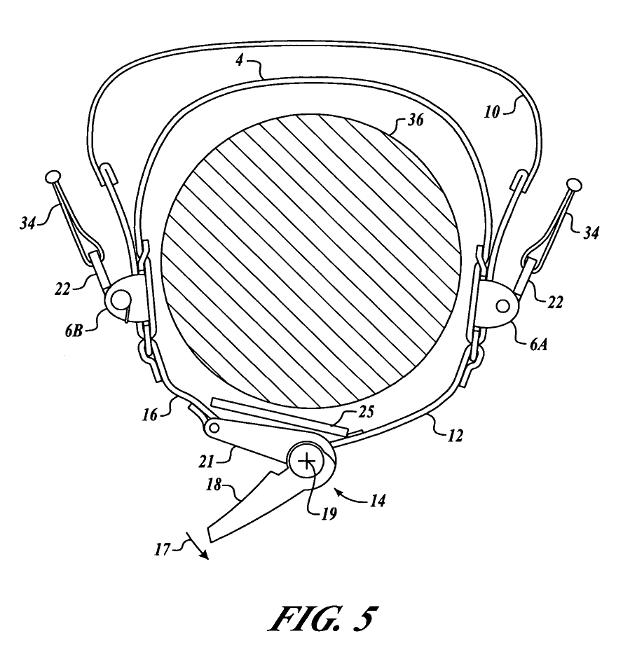

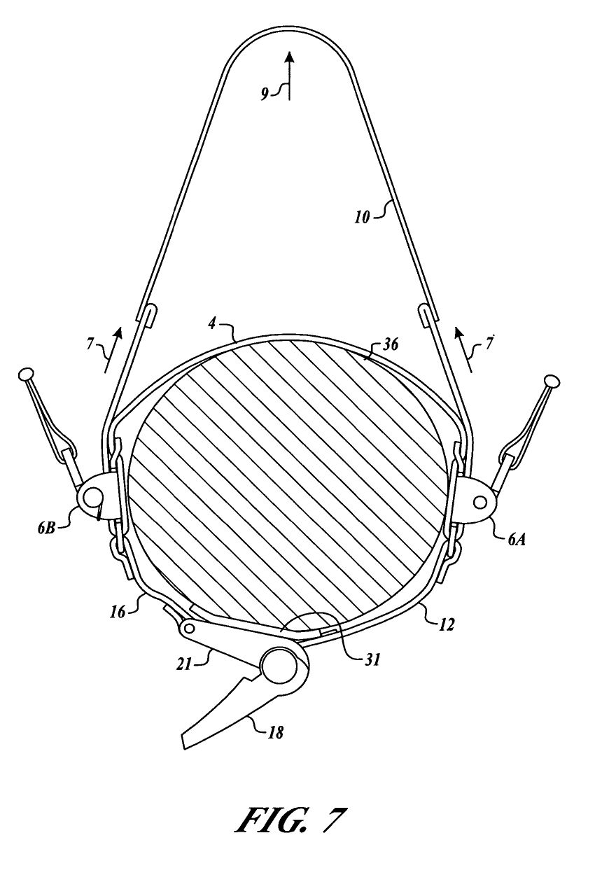

Referring to FIGS. 1 through 5, the tourniquet device of this invention as illustrated is generally designated 2. The invention has a first belt 4 having opposite ends 8 which loop back through respective “slip” buckles, 6A and 6B, which are so called because they each allow the belt 4 to easily slip through in one direction, indicated by arrow 7, but prevent movement of the belt through said each in the opposite direction. The first belt 4 functions as one of two adjustable length segments of a tourniquet loop of this invention. A pull cord 10 extends between the first belt's opposite ends, each end of the pull cord 10 being connected to a respective end 8 of the first belt 4. In operation, pulling the cord 10 (in the general direction indicated by arrow 9 in FIG. 7) draws the end portions of the first belt 4 further through respective slip buckles 6A and 6B, in directions indicated by the arrows 7, and thereby shortens the segment of the first belt 4 disposed between the slip buckles 6A and 6B. Alternatively, the pull cord 10 can be an integral extension of the first belt 4 in which case the first belt 4 would be a complete loop.

The pull cord 10 may also comprise one or more cords extending from either or both ends 8 of the first belt 4. For example, a single cord 10 may extend from the end 8 of the first belt 4 on the left hand side of FIG. 1 without extending all the way to the end 8 of the first belt 4 on the right hand side of FIG. 1. Alternately, the single cord 10 may extend from the end 8 of the first belt 4 on the right hand side of FIG. 1 without extending all the way to the end 8 of the first belt 4 on the left hand side of FIG. 1. In yet another aspect, the single cord 10 may comprise two separate cords extending from both the end 8 of the first belt 4 on the right hand side of FIG. 1 and the end 8 of the first belt 4 on the left hand side of FIG. 1.

Referring again to FIGS. 1 through 5, a second belt 12, which is the second of the two adjustable length segments of the tourniquet loop of this invention, has one end connected to a belt-coupling loop 11A extending from one of the two slip buckles 6A while the second belt's other end is engaged with a belt tightening device, generally designated 14. The belt tightening device 14 preferably includes a cylindrical spool 20 to which the other end of the second belt 12 is affixed for being spooled, a lever 18, and a ratchet-action mechanism (not shown) which rotates the spool 20 in a spooling direction whenever a user operates the lever 18. The lever 18 is operated by reciprocally rotating the lever 18 about the ratchet mechanism's axis 19. In operation, a user selectively shortens the free length, i.e. the unspooled length, of the second belt 12 by operating the lever 18 to reel more of the second belt 12 onto the spool 20. The belt tightening device 14 further includes an elongated arm 21 which extends from the ratchet mechanism and has a distal end which is connected to a belt-coupling loop 11 extending from the other slip buckle 6B, the connection being preferably by means of a relatively short, fixed-length, third belt 16. In operation, the elongated arm 21 is actually a segment of the tourniquet loop and when the loop is coarsely tightened about a limb, as will be explained further below, the tautness of the loop sufficiently stabilizes the elongated arm 21 to enable it to react against the torque applied to the ratchet mechanism by the lever 18 during its operation. The third belt 16 can alternatively be replaced by any other suitable means for connecting the distal end of the elongated arm 21 to the tourniquet loop.

In operation, by pulling the lever 18 away from third belt 16 in the direction indicated by arrow 17, the second belt 12 will be further spooled by one or more ratchet increments depending on the angular stroke of the levers 18. When the lever 18 so pulled, a pair of ratchet gears (not shown) at opposite ends of the spool 20 are rotated by the lever, and as the ratchet gears rotate, a pair of spring-biased detents (not shown) ride on the gears' teeth and are urged to seat in the valleys between adjacent teeth to prevent unspooling of the second belt 12 as the lever is reciprocated backward for another pull, i.e., the detent catches keep the second belt 12 spooled while allowing the lever 18 to be pushed back to its starting point for additional pulls if desired. As the lever 18 is repeatedly operated in pump fashion, the detents will continue to ride on and catch the ratchet gears' teeth until the unspooled segment of second belt 18 has been shortened to a user selected extent. Preferably the angular stroke of the lever 18 in cooperation with the ratchet mechanism allows a user to spool through multiple ratchet increments for each pull so that the free segment of the second belt 12 can be rapidly shortened if desired.

Referring to FIGS. 2-4, each slip buckle 6A and 6B has a manually operated, spring-biased, release lever 22 which projects preferably tangentially from an integral cylindrical cam 26 having a longitudinal eccentricity having a ridged surface illustrated here as a plurality of longitudinal rows of teeth 28 projecting outwardly, generally normal to the lever 22. The cam 26 pivots on a pair of integral pintles 24 projecting oppositely from the cam's ends, the pintles 24 being turnable in mating holes defined by lateral (with respect to belt 4) opposing flanges 25 projecting normally from a planar buckle base 27, and extending longitudinally from the buckle base at opposite ends are belt guide loops, 11A and 11B. As best illustrated in FIGS. 2 and 3, a respective end portion of the first belt 4 extends through the guide loop 11B of each slip buckle 6A and 6B to pass underneath the buckle's base 27, then extends up through guide loop 11A, then folds back to extend over the buckle base 27 and beyond, out of the slip buckle 6A and 6B. For each slip buckle 6A and 6B, a coiled spring 30 encircles about one of the buckle's pintles 24. The spring 30 has one end (not shown) disposed in a pintle hole (not shown) defined in the pintle 24, and the spring's other end is anchored by the closest flange, and is held in place by a pintle cap 31. The spring 30 biases the angular disposition of the cam 26 so that the cam's eccentricity impinges, i.e. presses against the belt 4, wedging the belt 4 between the eccentricity's ridged surface 28 and the buckle's base 27. Thus, pulling the first belt 4 in a direction opposite to that of arrow 7 causes the belt 4 to be seized and wedged by the eccentricity, and the belt 4 will not move. Whereas, pulling the belt 4 in the direction of arrows 7 will meet no such resistance. Moreover, pivoting the release lever 22 against the bias, i.e. in the direction indicated by arrow 23, rotates the eccentricity away from the first belt 4 and releases the belt 4 to be moved freely through the slip buckle 6A and 6B, allowing the belt 4 to be pulled through the slip buckle 6A and 6B in either direction. Preferably, a lanyard 34 is looped through a hole 29 defined proximate the end of the release lever 22 to make it easier to pull the release lever 22 against the bias. By the use of the levers 22, a user can readily expand the length of the first belt's segment of the tourniquet loop to widen the loop prior to application or to quickly release the tourniquet loop.

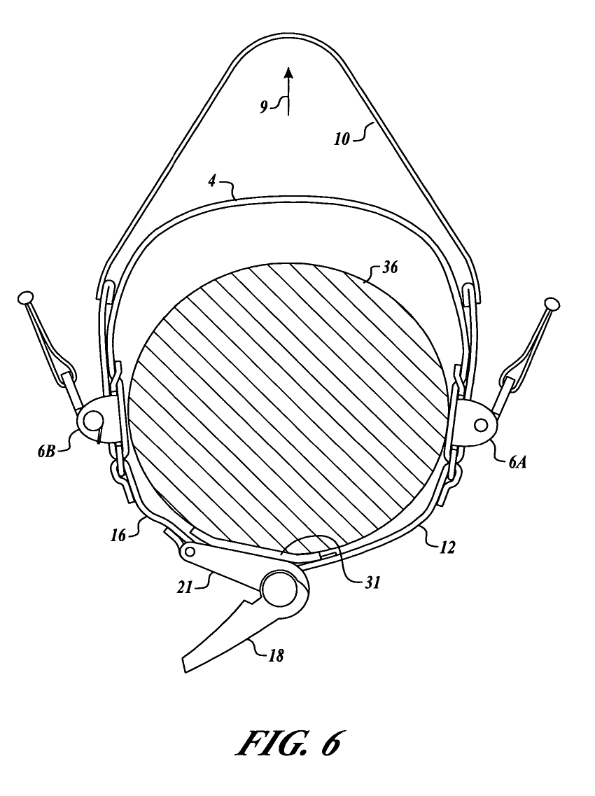

Referring to FIG. 5, prior to applying this invention to a limb 36, the tourniquet loop is preferably expanded sufficiently to be easily slipped over the limb 36. Expansion of the loop is preferably done by increasing the tourniquet loop segment lengths of both the first and second belts, 4 and 12, respectively. The belt tightener 14 further includes a release mechanism (not shown) by which a user can release the ratchet mechanism and easily unspool the second belt 12 to expand its segment of the tourniquet loop. Expansion of the first belt's segment of the tourniquet loop is accomplished by pulling the release lever 22 of each slip buckle away from the first belt 4 in the direction indicated by arrow 23 (of FIG. 2), and then pulling the first belt through said each slip buckle 6A and 6B in a direction opposite to that indicated by arrows 7 (of FIG. 2).

Referring to FIGS. 5-8, the preferred operation of this invention is described in terms of self-application. As a first step, a person having a severely bleeding limb 36 slips this invention over the limb so that the tourniquet loop loosely circumscribes the limb 36, as best illustrated in FIG. 5. Preferably, the tourniquet loop is positioned on the limb 36 to allow easy access to the lever 18 of the tightener 14. Also, preferably, a pad 31 is disposed between the limb 36 and the belt tightener 14 to avoid any discomfort which may be caused by the tightener 14 and/or its operation, such as a possible pinching of flesh. Preferably the pad 31 is of resilient material such as compressible foam. After the tourniquet loop loosely circumscribes the limb, the user begins pulling on cord 10 (FIG. 6) in the general direction of arrow 9 to cause the second belt 12, the third belt 16, and/or the arm 21 of the belt tightener 14 to generally abut the limb 36. Stronger pulling of cord 10 in the direction of arrow 9, as best illustrated in FIG. 7, will result in a significant shortening of the first belt 4 segment of the tourniquet loop until the tourniquet loop is coarsely tightened about the limb 36. Once the tourniquet loop is coarsely tightened about the limb 36, the loop is tense enough to sufficiently stabilize the ratchet arm 21 to allow a user to operate the belt tightener's lever 18 to finely reel-in the second belt 12 onto the tightener's spool 20 to a point at which a medically appropriate tourniquet has been achieved.

To loosen the invention for removal from a limb, the lanyards 34 can be pulled causing the release levers 22 to release their respective cams' grip on the first belt 4. Pulling on the first belt 4 will pull the first belt 4 through the slip buckles 6A and 6B, loosening the device on the limb 36. Alternately, the ratchet mechanism can be released and the second belt 12 unspooled somewhat to temporarily release the tourniquet while keeping the tourniquet coarsely taut enough to reapply the tourniquet by operation of the belt tightener lever 18.

In addition to being useful as a tourniquet, the invention may also be employed as a pressure device to isolate and apply pressure to a specified area. In such a use, the tourniquet device 2 can be placed over dressings and tightened as described above, creating a pressure bandage. If desired, the tourniquet device 2 can also be used in the absence of a dressing in order to apply pressure to a desired area by placing it over that area and tightening in the above described manner.

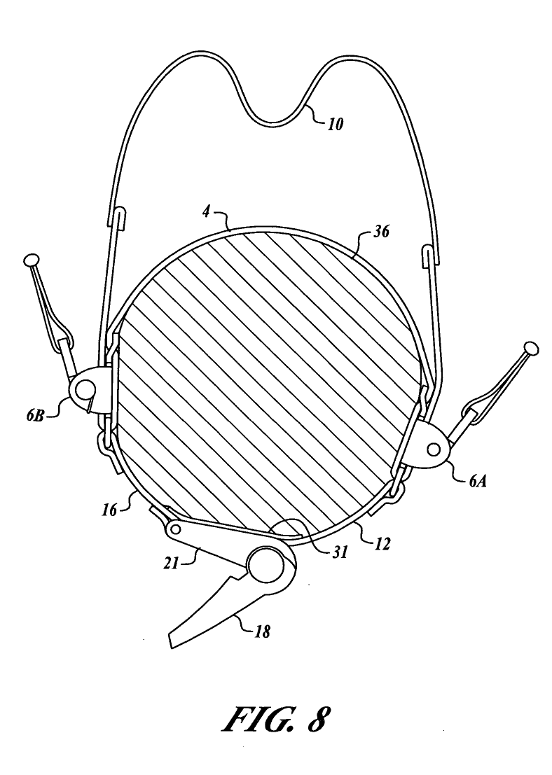

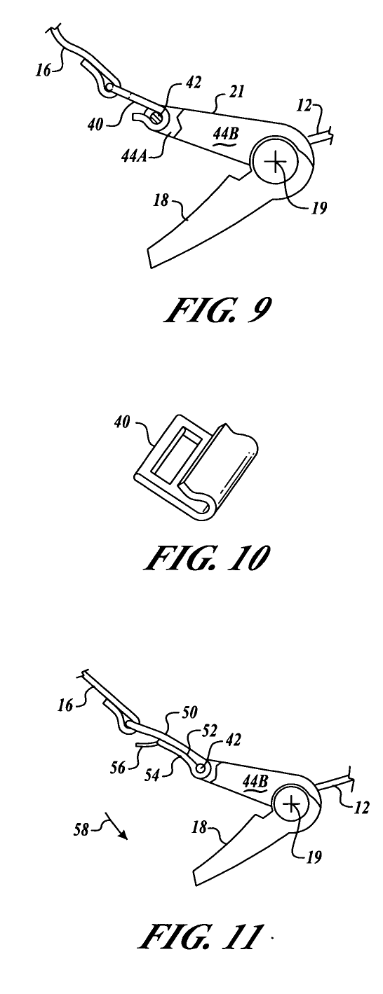

Referring to FIGS. 9-10, an alternative embodiment is illustrated in which the tourniquet loop can be opened and closed to facilitate use of the device. For example, the loop can be opened to apply it to a limb and then re-closed to tighten it around the limb, preferably by means of a detachable link in the tourniquet loop. Although the detachable link can be disposed anywhere around the tourniquet loop, a preferred embodiment is illustrated as a hook clasp 40 disposed between one of the slip buckles and the belt tightener. The hook clasp 40 is affixed to the end of the belt 16 remote from slip buckle 6B, and can be selectively hooked or unhooked onto a pin 42 extending between the distal ends of opposing elongate plates, 44A and 44B, which form the belt tightener's elongated arm 21.

FIG. 11 illustrates another preferred embodiment in which the detachable link comprises a reusable hook and loop fastener 50 such as and without limitation, Velcro®. In this embodiment, the fastener 50 can be quickly hooked together by pressing the opposing surfaces 52 and 54 of the fastener 50 together. In addition, the fastener 50 can be quickly detached by pulling an access tab 56 in direction 58.

In addition to the hook clasp 40 and the reusable fastener 50 mentioned above, other possible fasteners such as hook and eye fasteners or any other suitable fastener known in the art can also be employed as detachable links.

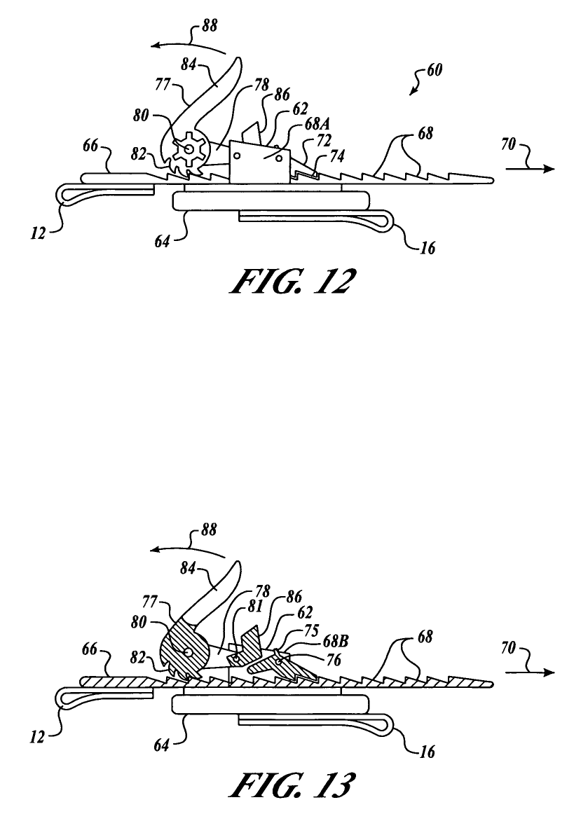

Another preferred embodiment of the belt tightening device 14 of the invention is illustrated in FIGS. 12 and 13. In FIG. 12 a plan view of a sliding slip buckle 60 is provided. FIG. 13 shows this same sliding slip buckle 60 in cross-section.

As illustrated, a buckle frame 62 is affixed to a buckle anchor 64, with the third belt 16 of the tourniquet being attached to the buckle anchor 64. It should be noted that the buckle anchor 64 can be omitted entirely with the third belt 16 being attached directly to the buckle frame 62. Though not shown for the sake of pictorial clarity, a comfort pad may be included between the sliding slip buckle 60 and the limb to which the tourniquet is being applied. This pad rests on the third belt 16 (and the buckle anchor 64, if present) and protects against discomfort, such as pinching of the flesh, which might otherwise be caused by applying the tourniquet to a limb.

Still referring to FIGS. 12 and 13, a rigid ratchet tab 66, to which the second belt 12 is attached, is provided to slide through an open space existing between opposing walls 68A and 68B of the buckle frame 62. The ratchet tab 66 includes a plurality of ridges 68, which allow the tab 66 to slide in direction 70 while a gripping ratchet 72 is engaged to contact the ridges 68 on the tab 66. In contrast, corresponding protrusions on a contact surface 74 of the gripping ratchet 72 preclude the tab 66 from moving in a direction opposite to the direction 70 while the gripping ratchet 72 is engaged. The gripping ratchet 72 is biased to contact the tab 66 by a spring 75 coiled around a support pin 76, with the support pin rotatably attaching the gripping ratchet 72 to the buckle frame 62.

An adjustment lever 77 is rotatably attached to the buckle frame 62 by a cross member 78 and pins 80 and 81. The adjustment lever 77 has a plurality of gripping extremities 82 formed on a contact surface capable of being placed in contact with tab 66. A gap 84 exists in an upper portion of the adjustment lever 77 to allow the protrusion of a release lever 86 there through. The release lever 86 is rotatably attached to the buckle frame 62 by pin 81.

In operation, when the adjustment lever 77 is pulled in direction 88, the extremities 82 contact the ridges 68 on the ratchet tab 66, propelling the tab 66 in direction 70. In this way, a user can tighten the tourniquet by moving belt 16 closer to belt 12. A spring (not shown) biases the adjustment lever 77 to rotate in a direction opposite to direction 88.

In order to loosen the tourniquet and move the tab 66 in a direction opposite to direction 70, a user need only press the release lever 86 towards the tab 66. In so doing, the release lever 86 contacts the gripping ratchet 72 and forces it to rotate about pin 76 counter to the biasing force of spring 75 such that the contact surface 74 of the gripping ratchet is released from contacting the ridges 68 of the pad 66. Once this is accomplished, the tab 66 is free to slide in direction 70 as well as a direction opposite thereto.

It should be noted that in addition to the buckle 60 discussed in conjunction with FIGS. 12-13, and the cylindrical spool 20 with level 18 and ratchet-action mechanism discussed in conjunction with FIGS. 1-10, any other locking ratchet device known in the art may be used with the invention.

The foregoing description and drawings were given for illustrative purposes only, it being understood that the invention is not limited to the embodiments disclosed, but is intended to embrace any and all alternatives, equivalents, modifications and rearrangements of elements falling within the scope of the invention. For example the two adjustable length segments of the tourniquet loop of this invention can be alternatively disposed such that the second belt, the spoolable segment, is disposed as a segment of the first belt intermediate the two slip buckles, without departing from the scope of this invention and the claims which follow. Also, one or both of the slip buckles can be integral with the spooling mechanism without departing from the scope of this invention and the claims which follow.

1. A tourniquet configured for single-handed application, the tourniquet comprising:
a cinching member, the cinching member having a first and a second attachment point and including:
a belt member, the belt member being a strap having a length between a securing end and a fly end, the secured end;
at least one slip buckle, the slip buckle having a body including the second attachment point, the body defining a tunnel, the tunnel configured to slidingly receive the belt member and further configured to selectively seize the belt member along the length, dividing the length into an outboard band and an inboard band, the outboard band being the length between the slip buckle and the fly end and the inboard band being the length between the slip buckle and the secure end; and
a ring member, the ring member including the first attachment point configured to encircle the belt member at the inboard band; and
a ratchet assembly, the ratchet assembly being secured to the cinching member between the first and the second attachment points.

2. The tourniquet of claim 1, wherein the cinching member is configured to allow drawing of the belt member slidingly through the slip buckle by tension on the outboard band.

3. The tourniquet of claim 2, wherein the fly end is secured to the securing end to form a loop.

4. The tourniquet of claim 1, wherein the slip buckle includes a seizing lever, the seizing lever having a seized position and a released position such that in the seized position the slip buckle seizes the belt at a point along the length and in the released position, the slip buckle slides freely along the length.

5. The tourniquet of claim 1, wherein the ring member includes a second slip buckle including a second seizing lever and configured such that tension on the outboard band draws the slip buckle and the second slip buckle together on the inboard band.

6. The tourniquet of claim 1, wherein the outboard band is substantially larger than the inboard band.

7. The tourniquet of claim 1, wherein a ratchet assembly includes:
a strap having a root end, a tongue end, a principal axis extending from the tongue end to the root end and at least a top surface and a bottom surface, the root end secured to the body substantially at the first attachment point, the top surface;
a housing adapted to slidably receive the tongue end, the housing being secured to the second end substantially at the second attachment point;
a pawl pivotably mounted to the housing about a first axis, having an engagement end and a disengagement end, each end located on opposing sides of the first axis, wherein the pawl is pivotably biased with a biasing means such that the engagement end is biased to rotate in an engagement direction about the first axis.
a rotatable spool mounted to the housing about a second axis, the second axis being substantially parallel to the first axis, the spool being substantially cylindrical having a first and a second substantially circular side and a face, the being configured to engage the top side of the strap; and
a spool gear mounted on the first substantially circular side of the spool, the spool gear configured to engage the pawl at the engagement end thereby allowing the spool to rotate about the second axis in a tightening direction and preventing the spool to rotate about the second axis in a loosening direction.

8. The tourniquet of claim 7, wherein the spool gear includes a spool-advancing lever having one end releasably attached to the spool gear, the spool-advancing lever configured for rotating said spool about the second axis in the tightening direction.

9. The tourniquet of claim 7, wherein the pawl includes a spool release lever configured to pivot the pawl in the disengagement direction, thereby removing the engagement end from engagement with the spool gear.

10. The tourniquet of claim 7, wherein the top surface includes a series of ridges substantially perpendicular to the principal axis and wherein the face is configured to releasably engage at least one of the ridges.

11. The tourniquet of claim 7, wherein the strap is removable from the housing.

12. A device for single-handedly applying a tourniquet loop to a limb comprising:
a first and a second slip buckle;
a tourniquet loop, the tourniquet loop comprising an outboard band and an inboard band and entraining the first and a second slip buckles in opposed relationship such that tension on the outboard band will draw the first and second slip buckles together on the inboard band; and
a ratchet device having a first end secured to the first slip buckle and a second end secured to the second slip buckle.

13. The device according to claim 12 wherein the inboard band includes a link, the link configured to be opened and closed as needed.

14. The device according to claim 12 wherein the outboard band includes a handle, the handle being gripable by a single hand, the handle configured for pulling the tensioning the outboard band drawing the tourniquet loop through the slip buckles in respective directions in order to shorten the inboard band between the slip buckles.

15. The device according to claim 14 wherein at least one slip buckle further comprises a release which when selectively actuated allows the first belt to be pulled through said at least one slip buckle in either direction.

16. The device according to claim 12 wherein at least one slip buckle further comprises:
a base;
a cylinder rotatable on an axis lateral to the inboard band, the cylinder including a ridged eccentricity, the cylinder axis being so disposed in fixed relation to the base that the eccentricity can be rotated in an engaging direction to impinge upon the inboard band extending between the base and the cylinder and when so rotated the eccentricity seizing and wedging the inboard band against the base; and
a bias which urges the cylinder in the engaging direction.

17. The device according to claim 16, wherein the slip buckle further comprises a lever by which a user can rotate the cylinder in a disengaging direction against the bias to release the first belt.

18. A method for impeding the flow of blood in a limb, the method comprising:
encircling the limb with a tourniquet device, the tourniquet device including a cinching device and a ratchet device;
cinching the cinching device tensioning the tourniquet device to bear against the limb; and
ratcheting the ratcheting device to suitable tension the tourniquet device to impede the flow of flow of blood.

19. The method of claim 18, wherein cinching includes:
drawing a belt through the cinching device; and
locking the belt in engaged relationship to the cinching device to suitably tension the tourniquet device.

20. The method of claim 18, wherein encircling the limb includes engaging the ratchet device, that ratchet device including a tongue and a spool, the tongue configured to releasably engage the spool.

21. The method of claim 20, wherein ratcheting includes operating a ratcheting lever, the ratcheting lever configured to rotate the spool in a drawing direction, to suitably tension the tourniquet device.
